(19) United States
(12) Patent Application Publication
(10) Pub. No.: US 2015/0025574 A1
(43) Pub. Date: Jan. 22, 2015

(54) OCCIPITAL PLATE

(71) Applicant: John Mackall, Arlington, VA (US)

(72) Inventor: John Mackall, Arlington, VA (US)

(21) Appl. No.: 14/506,894

(22) Filed: Oct. 6, 2014

Related U.S. Application Data
(63) Continuation-in-part of application No. 13/429,203, filed on Mar. 23, 2012.

(51) Int. Cl. A61B 17/70

(52) U.S. Cl. CPC A61B 17/7055; A61B 17/7059
USPC 606/246

(57) ABSTRACT

An occipital plate for use in an occipitocervical fixation procedure to stabilize the base of a patient's skull with respect to the patient's neck. The occipital plate is made up of a middle portion having left and right sides, and left and right hinged legs extending outward in opposite directions from the left and right sides of the middle portion. Each of the left and right hinged legs uses a hinge mechanism to secure a spinal rod to the occipital plate.

### PRIORITY

This application claims priority as a Continuation-in-part Application under 35 U.S.C. §120 to U.S. Non-provisional Patent Application entitled “Occipital Plate,” filed in the U.S. Patent and Trademark Office on Mar. 23, 2012, and assigned Ser. No. 13/429,203, which is incorporated in its entirety herewith.

### FIELD OF THE INVENTION

The embodiments relate to medical devices. More specifically, the embodiments are directed to an occipital plate.

### BACKGROUND OF THE INVENTION

As noted in U.S. Pat. No. 7,695,500 issued to Markworth, there are many occipital plate implants on the market today. Some implants have through-holes and must be preloaded on the rod. Others have top loading sockets similar to polyaxial screws that allow bent rods to be anchored to the plate. The most simple, but generally most difficult, to use form is that of a rod that smoothly tapers to an occipital plate that must be bent and contoured to match highly varied anatomy. All of these iterations have at most one-to-two degrees of freedom and typically require long preparation time to ensure a proper bend.

This makes the implants both difficult to connect to the longitudinal rod member and the occipital plateau without putting stress on the atlantoaxial joint.

Accordingly, there remains a need for an improved occipital plate.

### SUMMARY OF THE INVENTION

An object of the embodiments is to substantially solve at least the problems and/or disadvantages discussed above, and to provide at least one or more of the advantages described below.

It is therefore a general aspect of the embodiments to provide an occipital plate that will obviate or minimize problems of the type previously described.

According to an aspect of the embodiments, an occipital plate for use in an occipitocervical fixation procedure to stabilize the base of a patient's skull with respect to the patient's neck is provided herein. The occipital plate according to aspects of the embodiments is made up of a middle portion having left and right sides, and left and right hinged legs extending outward in opposite directions from the left and right sides of the middle portion. Each of the left and right hinged legs uses a hinge mechanism to secure a spinal rod to the occipital plate.

According to a first aspect of the embodiments, an occipital plate is provided comprising a middle portion having opposite left and right sides, a left rounded locking hinge for receiving and securing a first spinal rod (SR1), wherein the left rounded locking hinge extends from the left side of the middle portion, and wherein the left rounded locking hinge includes a left leg middle portion that is concavely curved with respect to a left side substantially flat surface of the left side of the middle portion, and a left leg distal protrusion located at a distal end of the left leg middle portion, and a right rounded locking hinge (2408) for receiving and securing a second spinal rod (SR2), wherein the right rounded locking hinge extends from the right side of the middle portion, and wherein the right rounded locking hinge includes a right leg middle portion that is concavely curved with respect to a right side substantially flat surface of the right side of the middle portion, and a right leg distal protrusion located at a distal end of the right leg middle portion.

According to the first aspect of the embodiments, the middle portion further comprises a left side shaped receptacle adapted to receive the left leg distal protrusion located at the distal end of the left leg middle portion when the left rounded locking hinge is rotated to secure the first spinal rod, and a right side shaped receptacle adapted to receive the right leg distal protrusion located at the distal end of the right leg middle portion, when the right rounded locking hinge is rotated to secure the second spinal rod.

According to the first aspect of the embodiments, the left leg distal protrusion, the right leg distal protrusion, the left side shaped receptacle, and the right side shaped receptacle are shaped according to a shape that can include at least one of an oval, a circle, a square, triangle, and a rectangle, and further wherein the left shaped receptacle includes a middle portion left side receptacle lip adapted to retain the left leg distal protrusion, and the right shaped receptacle includes a middle portion right side receptacle lip adapted to retain the right leg distal protrusion.

According to the first aspect of the embodiments, the occipital plate further comprises a left side gripping surface located on the left side substantially flat surface adapted to grip said first spinal rod, a right side gripping surface located on the right side substantially flat surface adapted to grip said second spinal rod, a left rounded locking hinge gripping surface located on a surface of the left rounded locking hinge opposite to that of the left side gripping surface when in the securing position, and adapted to grip said first spinal rod, and a right rounded locking hinge gripping surface located on a surface of the right rounded locking hinge opposite to that of the right side gripping surface when in the securing position, and adapted to grip said second spinal rod.

According to the first aspect of the embodiments, the middle portion comprises a left member, a right member, and a top member, wherein said top member extends from an uppermost portion of the left member to an uppermost position of the right member, and wherein substantially all edges are rounded. Still further according to the first aspect of the embodiments, a radius of curvature for the rounding of substantially all of the edges ranges from about 0.5 mm to about 1.0 mm.

According to the first aspect of the embodiments, the middle portion further comprises a left leg protrusion receiving portion adapted to receive the left leg distal protrusion located at the distal end of the left leg middle portion when the left rounded locking hinge is rotated to secure the first spinal rod, and a right leg protrusion receiving portion adapted to receive the right leg distal protrusion located at the distal end of the right leg middle portion, when the right rounded locking hinge is rotated to secure the second spinal rod.

According to the first aspect of the embodiments, the left leg distal protrusion, the right leg distal protrusion, the left leg protrusion receiving portion, and the right leg protrusion receiving portion are shaped according to a shape that can include at least one of an oval, a circle, a square, triangle, and a rectangle.

According to the first aspect of the embodiments, the left leg distal protrusion includes a substantially planar left leg protrusion surface, the left leg protrusion receiving portion includes a substantially planar left leg protrusion receiving surface adapted to retain the left leg distal protrusion, the right leg distal protrusion includes a substantially planar right leg protrusion surface, and the right leg protrusion receiving portion includes a substantially planar right leg protrusion receiving surface adapted to retain the left leg distal protrusion.

According to the first aspect of the embodiments, the substantially planar left leg protrusion surface and the substantially planar left leg protrusion receiving surface can both be substantially smooth, and the substantially planar left leg protrusion surface and the substantially planar left leg protrusion receiving surface can both include a knurled surface.

According to the first aspect of the embodiments, one of the substantially planar left leg protrusion surface and the substantially planar left leg protrusion receiving surface can include a knurled surface, and the substantially planar right leg protrusion surface and the substantially planar right leg protrusion receiving surface can both be substantially smooth.

According to the first aspect of the embodiments, the substantially planar right leg protrusion surface and the substantially planar right leg protrusion receiving surface can both include a knurled surface, and one of the substantially planar right leg protrusion surface and the substantially planar right leg protrusion receiving surface can include a knurled surface.

According to the first aspect of the embodiments, the left leg protrusion receiving portion comprises a left leg protrusion receiving portion fillet edge with a height h1 extending above an upper surface of the middle portion.

According to the first aspect of the embodiments, the right leg protrusion receiving portion comprises a right leg protrusion receiving portion fillet edge with a height h1 extending above an upper surface of the middle portion.

According to a second aspect of the embodiments, an occipital plate is provided comprising a middle portion having opposite left and right sides, a left hinged leg for receiving and securing a first spinal rod, wherein the left hinged leg extends from the left side of the middle portion, a right hinged leg for receiving and securing a second spinal rod, wherein the right hinged leg extends from the right side of the middle portion, and a left leg curved protrusion located at a distal portion of the left hinged leg, and a right leg curved protrusion located at a distal portion of the right hinged leg.

### BRIEF DESCRIPTION OF THE DRAWINGS

The above and other objects and features of the embodiments will become apparent and more readily appreciated from the following description of the embodiments with reference to the following Figures, wherein like reference numerals refer to like parts throughout the various Figures unless otherwise specified, and wherein:

FIG. 1 illustrates an environmental perspective view of an occipital plate, according to an embodiment;

FIG. 2 illustrates a front view of the occipital plate of FIG. 1 but with left and right hinged legs in an open configuration;

FIG. 3 illustrates a front view of the occipital plate of FIG. 1 but with the left hinged leg in a closed configuration and the right hinged leg in a partially closed configuration;

FIG. 4 illustrates a front view of the occipital plate of FIG. 1 but with the left and right hinged legs in a closed configuration according to an embodiment;

FIG. 5 illustrates a rear view of the occipital plate of FIG. 4;

FIG. 6 illustrates a top side view of the occipital plate of FIG. 4;

FIG. 7 illustrates a bottom side view of the occipital plate of FIG. 4;

FIG. 8 shows a front view of an occipital plate with first and second hinged legs comprising high friction front surfaces, according to an embodiment;

FIG. 9 illustrates an exploded view of the occipital plate of FIG. 1, according to an embodiment;

FIGS. 10, 11, 12, 13, 14 and 15 illustrate various views of an occipital plate according to different aspects of the embodiments;

FIG. 16 illustrates Table 1 listing part numbers of the occipital plates of FIGS. 1-15, and 16-23;

FIGS. 17, 18, 19, 20, 21, 22 and 23 illustrates additional views of the occipital plate according to further aspects of the embodiments;

FIG. 24 illustrates a front perspective of view of occipital plate with rounded locking hinges according to an embodiment;

FIG. 25 illustrates a close-up side view of a left side rounded locking hinge and receptacle portion according to an embodiment;

FIG. 26 illustrates a further front perspective view of the occipital plate of FIG. 24 according to an embodiment;

FIG. 27 illustrates a side view of a conventional (non-curved) rotatable leg portion when securing a first spinal rod against a bottom portion of an occipital plate, and a side view of the left rotatable leg portion according to an embodiment securing a first spinal rod against a bottom portion of the occipital plate of FIG. 24 according to an embodiment; and

FIG. 28 illustrates a close-up side view of a left side rounded locking hinge and receiving portion according to an embodiment.

### DETAILED DESCRIPTION OF THE EMBODIMENTS

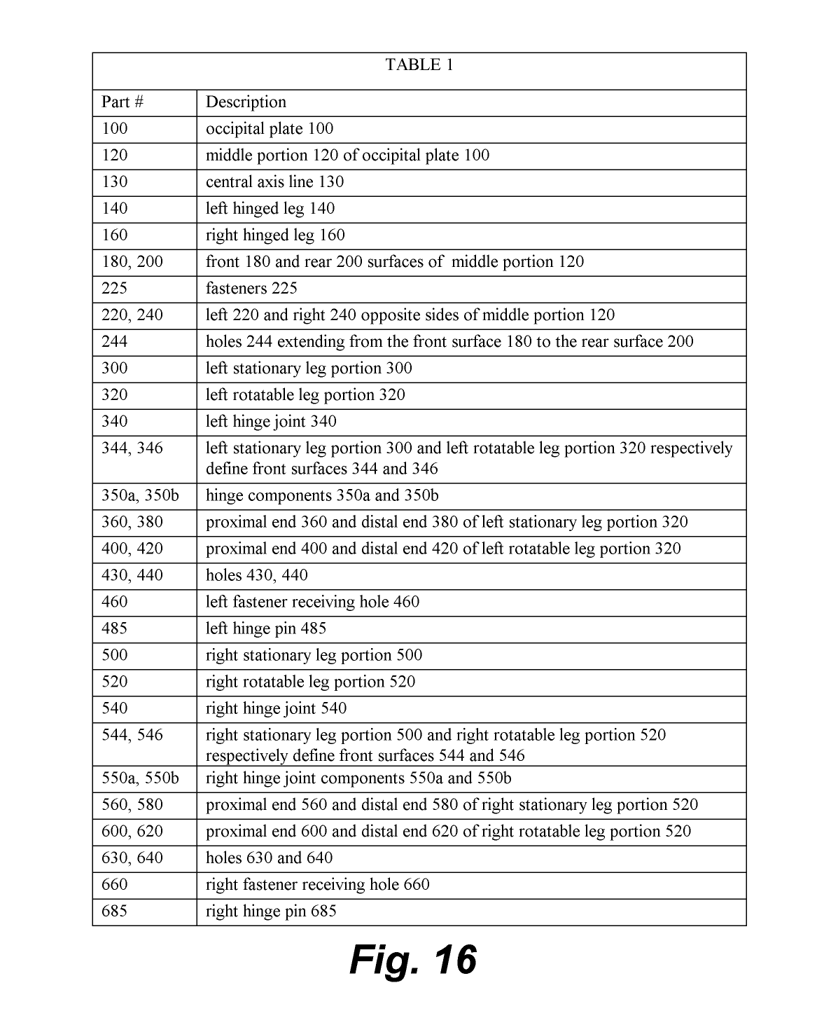

The embodiments described herein relate to medical devices. More specifically, the embodiments are directed to an occipital plate 100 for use in an occipitocervical fixation procedure. The occipital plate 100 and its parts can be made out of any suitable material such as, but not limited to, titanium, tungsten, and stainless steel, alone or in combination. Part numbers are listed in Table 1 (shown in FIG. 16).

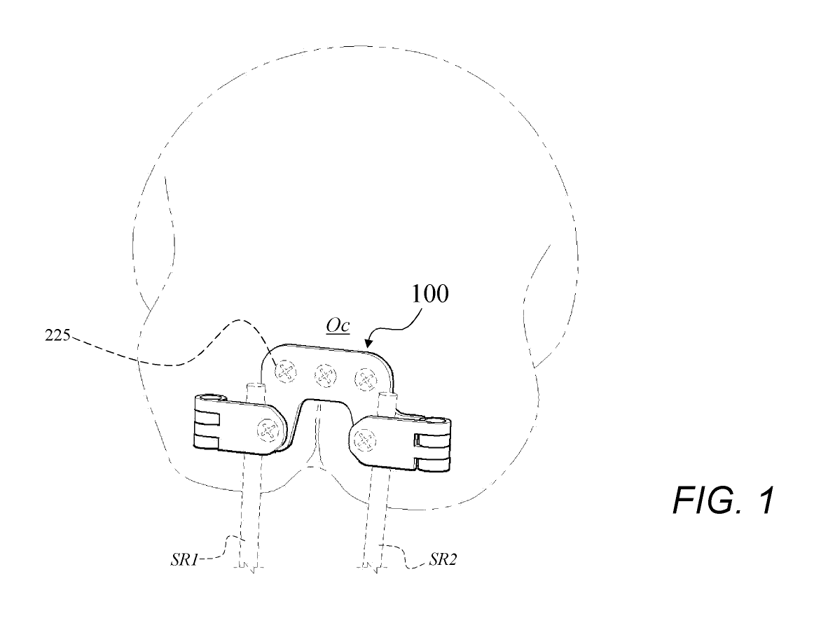

The occipital plate 100 comprises a middle portion 120, a left hinged leg 140, and a right hinged leg 160. The occipital plate 100 defines a central axis line 130. While not necessary, according to an embodiment, occipital plate 100 exhibits 2-fold symmetry about a central axis line 130. The middle portion 120 defines front 180 and rear 200 surfaces, and left 220 and right 240 opposite sides. Holes 244 extend from the front surface 180 through to the rear surface 200 of the middle portion 120 are provided for receiving bone fasteners 225 for fixation of occipital plate 100 to the occiput Oc, i.e., the posterior (back) portion of a patient's head (see FIG. 1). The holes 244 can be in the form of countersunk screw holes. The occipital plate 100 can be curved for facilitating attachment to the lower posterior curve of the skull known as the occipital region (labeled as “Oc” in FIG. 1).

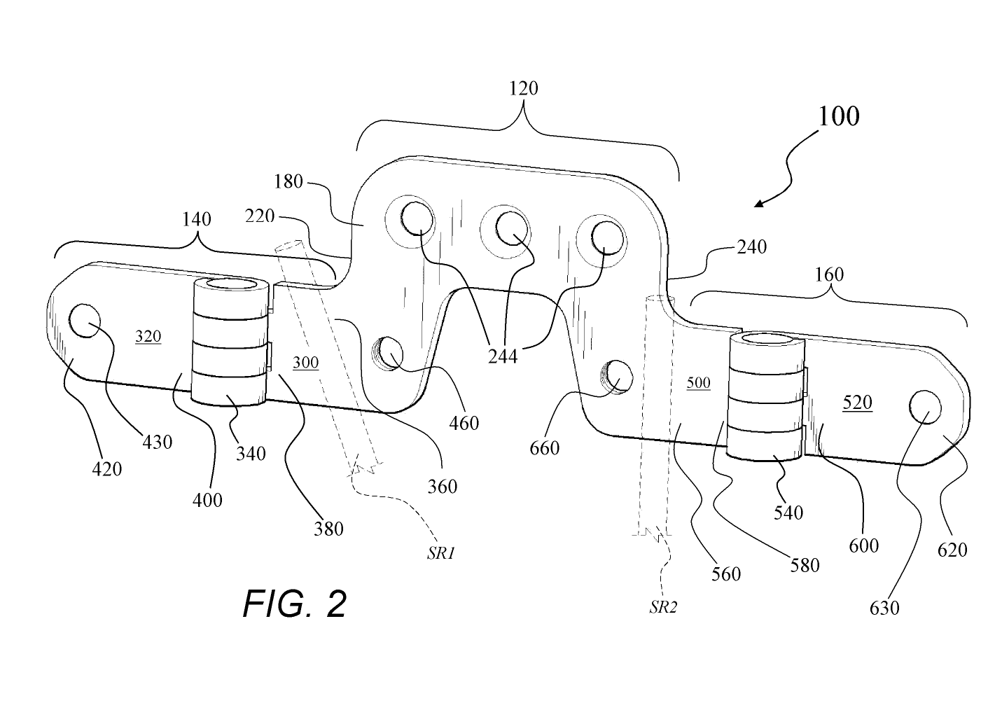

Left and right hinged legs 140 and 160 respectively extend outward from left 220 and right 240 opposite sides of the middle portion 120. The left hinged leg 140 comprises a left stationary leg portion 300, a left rotatable leg portion 320, and a left hinge joint 340. The left stationary leg portion 300 defines proximal end 360 and distal end 380 thereof. Left rotatable leg portion 320 defines proximal end 400 and distal end 420 thereof. The distal end 420 defines through-hole 430, which can be a countersunk screw hole. The terms “proximal” and “distal” are used with respect to the middle portion 120 when the hinged legs of occipital plate 100 are in an open configuration as shown in FIG. 2. The left hinge joint 340 can take any suitable form. According to an embodiment, the left hinge joint 340 comprises tubular hinge components 350a and 350b and left hinge pin 485.

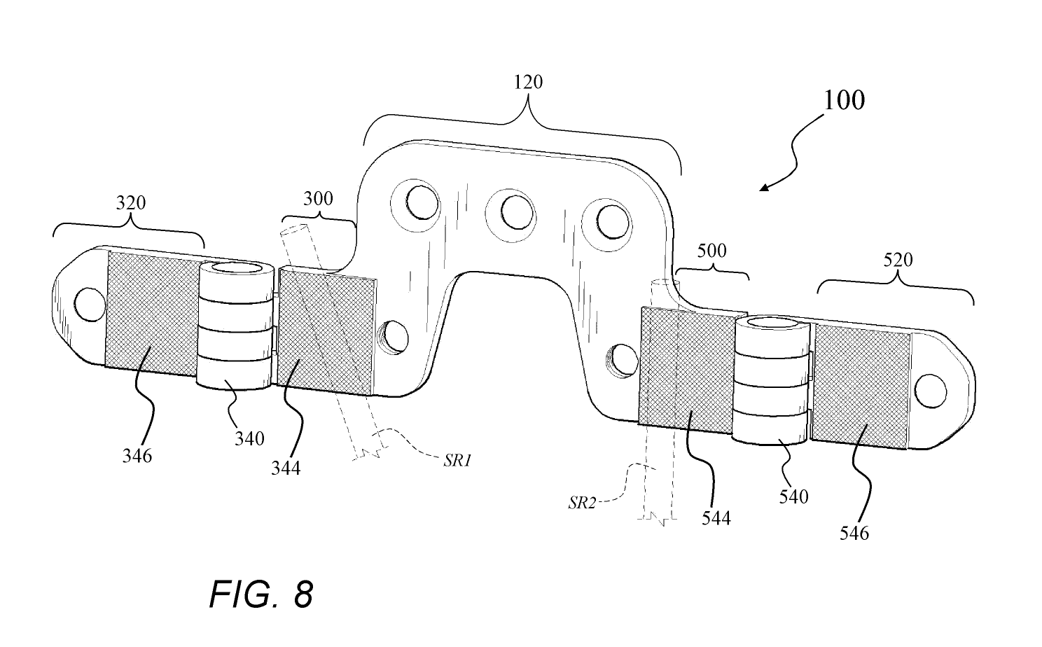

The left stationary leg portion 300 and left rotatable leg portion 320 are connected via the left hinge joint 340. More specifically, the distal end 380 of left stationary leg portion 300 and the proximal end 400 of left rotatable leg portion 320 are each connected to hinge joint 340. As shown, for example, in FIG. 2, the proximal end 360 of the left stationary leg portion 300 is integral with the left opposite side 220 of the middle portion 120 whereas the distal end 380 of the left stationary leg is operatively connected to the left hinge joint 340. The left rotatable leg portion 320 pivots about left hinge joint 340. The amount of rotation is sufficient to allow the securement of a spinal rod (represented by first spinal rod shown as “SR1” in FIG. 8) to the left hinged leg 140.

The term “front surface” refers to the surface that during and post-surgery is opposed to the one (the rear surface) that faces the bone of a patient's occipital region. The term “rear surface” refers to the surface that during and post-surgery faces the bone of a patient's occipital region. The left stationary leg portion 300 and left rotatable leg portion 320 respectively define front surfaces 344 and 346. The front surfaces 344 and 346 can be machined to provide high friction surfaces as shown in FIG. 8; according to an embodiment, such surfaces can be knurled, in order to bite into the surface of the spinal rod(s) (SR1 and SR2). As those of skill in the art can appreciate, other means for providing frictional surfaces can also be used, including, for example, but not limited thereto, tongue and groove assemblies, among others.

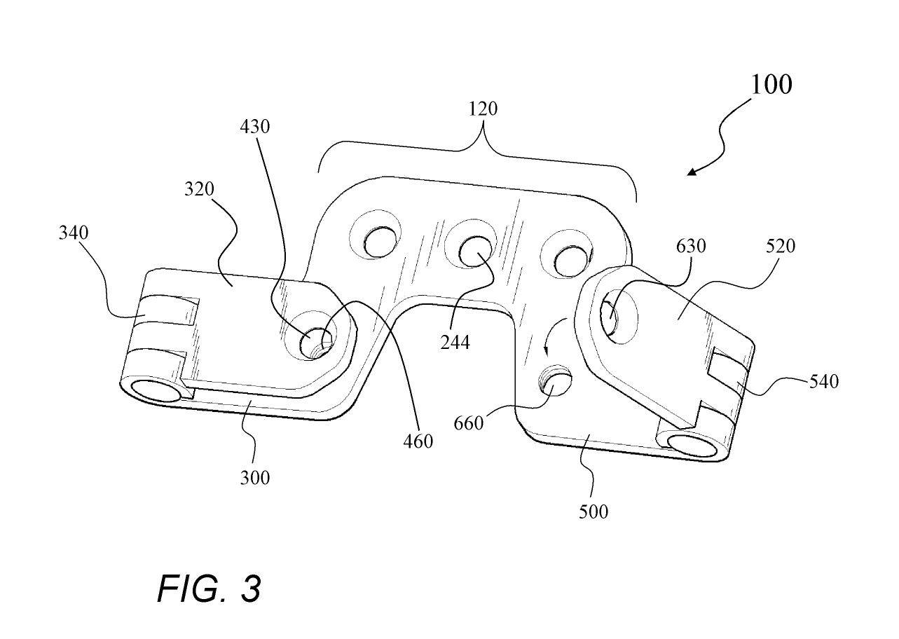

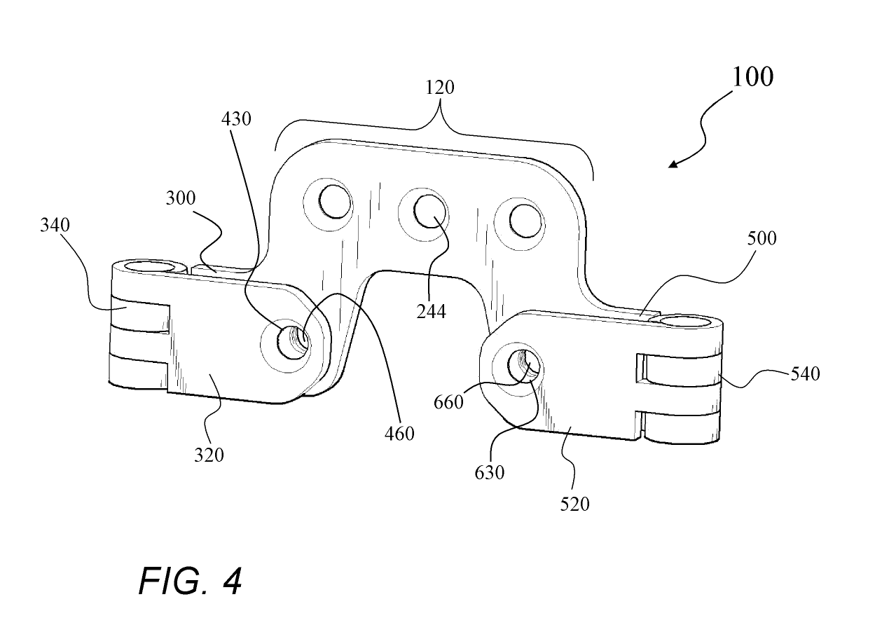

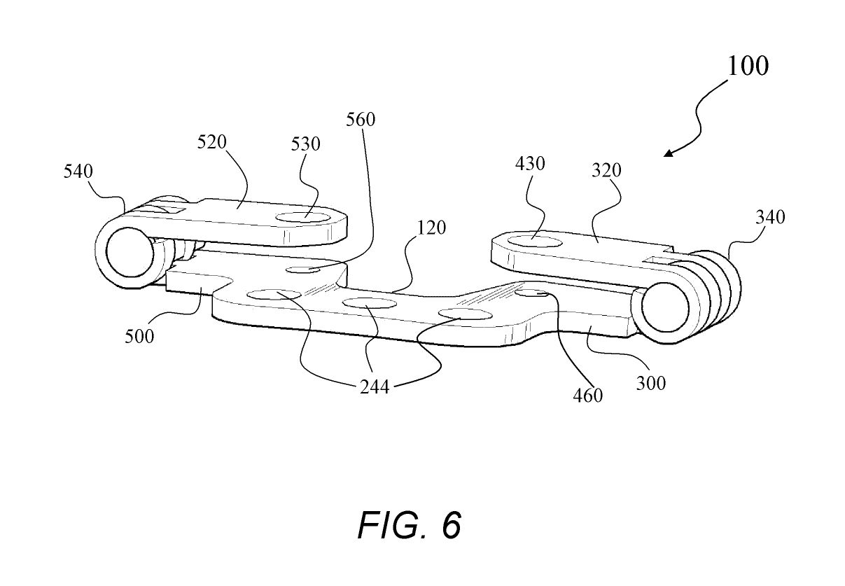

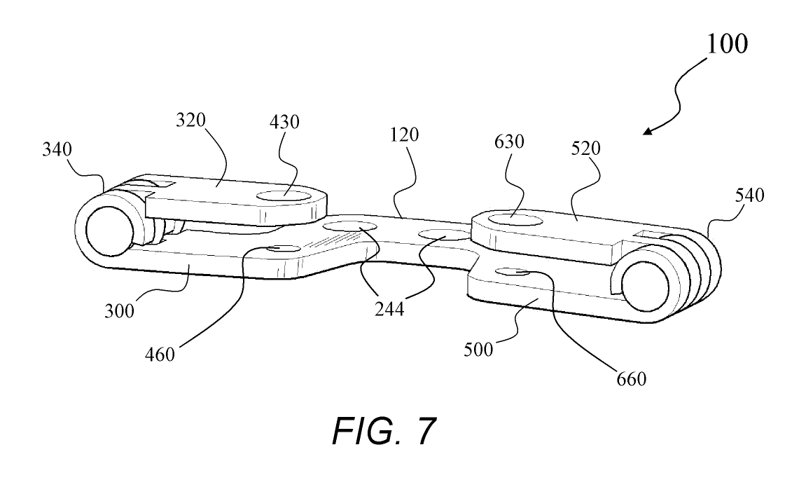

According to an embodiment, the left rotatable leg portion 320 of the left hinged leg 140 is of sufficient length to allow its distal end 420 to be directly fastened to the middle portion 120 of occipital plate 100 (see, e.g., FIGS. 2 through 7). In this embodiment, the middle portion 120 defines a complementary left fastener receiving hole 460. During surgery the left rotatable leg portion 320 is rotated about the hinge joint 340 until the through hole 430 of distal end 420 is aligned over the complementary left fastener receiving hole 460 whereupon a fastener, such as a locking screw, is affixed into hole 460 via through hole 430 to secure the left rotatable leg portion 320 to the middle portion 120. Fasteners are shown in FIG. 1.

The purpose of securing the distal end 420 of the left hinged leg 140 to the middle portion 120 is to clamp a spinal rod (represented by alpha-numeric label SR1 in, e.g. FIGS. 1 and 2) to the occipital plate 100. This procedure provides flexibility to the surgeon who is not limited to the specific location of a prior art seat as typically found on a prior art occipital plates. Moreover, rods can be secured at various angles to the left hinged leg 140 as shown in FIG. 2.

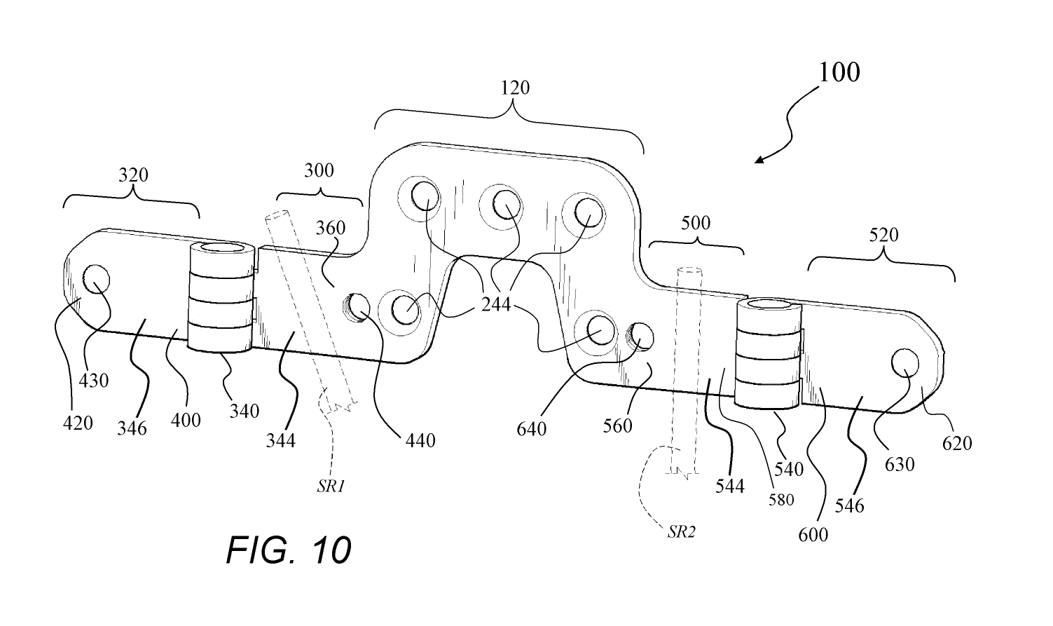

In another embodiment, the proximal end 360 of left stationary leg portion 300 defines a left fastener receiving hole 440 (see, FIG. 10). The fastener receiving hole 440 can be a threaded blind hole, or a non-threaded hole, as those of skill in the art can appreciate. In this embodiment a fastener, such as a locking screw, is used to fasten the distal end 420 of the left rotatable leg portion 320 to the proximal end 360 of stationary leg portion 300.

The right hinged leg 160 comprises a right stationary leg portion 500, a right rotatable leg portion 520, and a right hinge joint 540. The right stationary leg portion 500 defines proximal end 560 and distal end 580 thereof. Right rotatable leg portion 520 defines proximal end 600 and distal end 620 thereof. The proximal end 600 defines through-hole 630, which can be a countersunk screw hole. The terms “proximal” and “distal” are used with respect to the middle portion 120 when the hinged legs of occipital plate 100 are in an open configuration as shown in FIG. 2. The right hinge joint 540 can take any suitable form. In one embodiment, the right hinge joint 540 comprises tubular hinge components 550a and 550b and right hinge pin 685.

The right stationary leg portion 500 and right rotatable leg portion 520 are connected via the right hinge joint 540. More specifically, the distal end 580 of right stationary leg portion 500 and the proximal end 600 of right rotatable leg portion 520 are each connected to hinge joint 540. As shown, for example, in FIG. 2, the proximal end 560 of the right stationary leg portion 500 is integral with the right opposite side 220 of the middle portion 120, whereas the distal end 580 of the right stationary leg is operatively connected to the right hinge joint 540. The right rotatable leg portion 520 pivots about right hinge joint 540. The amount of rotation is sufficient to allow the securement of a spinal rod (represented by second spinal rod shown as “SR2” in FIG. 8) to the right hinged leg 160.

According to an embodiment, the right rotatable leg portion 520 of the right hinged leg 160 is of sufficient length to allow its distal end 620 to be directly fastened to the middle portion 120 of occipital plate 100 (see, e.g., FIGS. 2 through 7). In this embodiment, the middle portion 120 defines a complementary right fastener receiving hole 660. During surgery, the right rotatable leg portion 520 is rotated about the hinge joint 540 until the through hole 630 of distal end 620 is aligned over the complementary fastener receiving hole 660 whereupon a fastener, such as a locking screw, is affixed into hole 660 via through hole 630 to secure the right rotatable leg portion 520 to the middle portion 120.

The purpose of securing the distal end 620 of the right hinged leg 160 to the middle portion 120 is to clamp a spinal rod (represented by alpha-numeric label SR2 in, e.g., FIGS. 1 and 2) to the occipital plate 100. This procedure provides flexibility to the surgeon who is not limited to the specific location of a prior art seat as typically found on a prior art occipital plates. Moreover, rods can be secured at various angles to the right hinged leg 160.

According to an embodiment, the proximal end 560 of right stationary leg portion 500 defines a right fastener receiving hole 640 (see, FIG. 10). The fastener receiving hole 640 can be a threaded blind hole. In this embodiment, a fastener, such as a locking screw, is used to fasten the distal end 620 of the right rotatable leg portion 320 to the proximal end 560 of stationary leg portion 500.

The right stationary leg portion 500 and right rotatable leg portion 520 respectively define front surfaces 544 and 546. According to an embodiment, the front surfaces 544 and 546 can be machined to provide high friction surfaces as shown in FIG. 8; such surfaces are knurled in order to bite into the surface of a spinal rod (represented by alpha-numeric label “SR2” in FIG. 8).

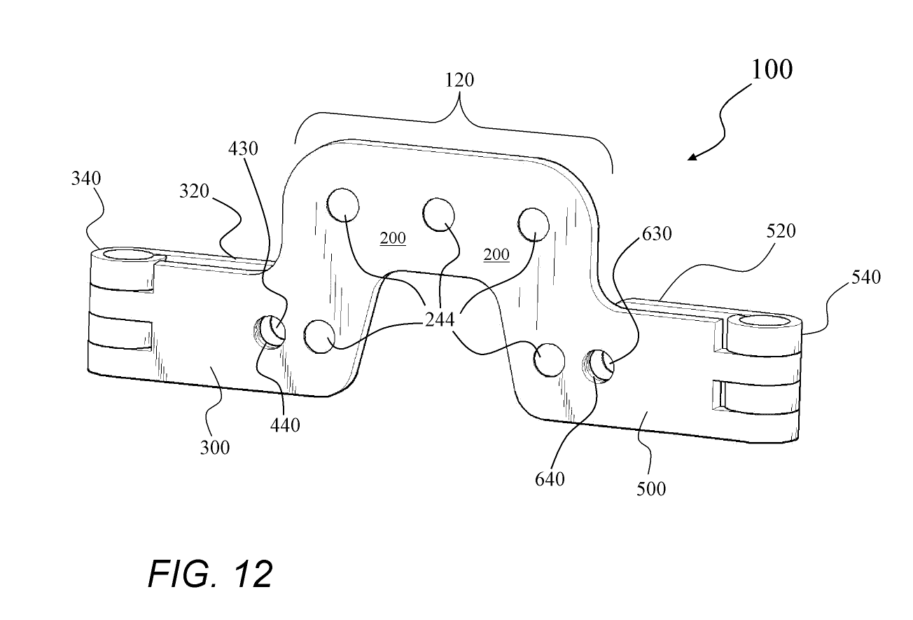

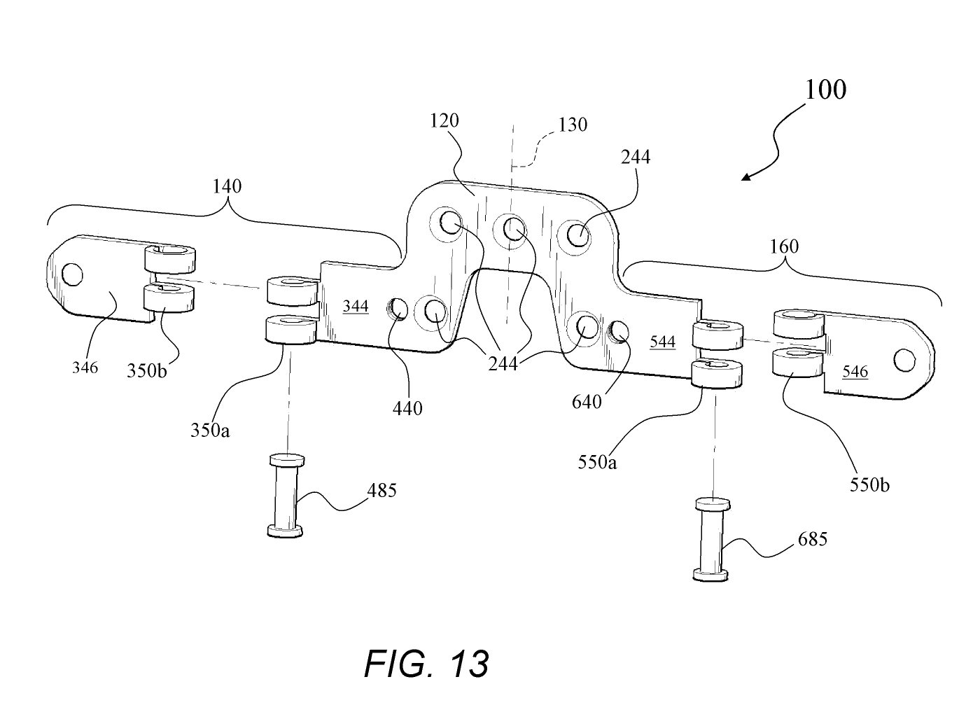

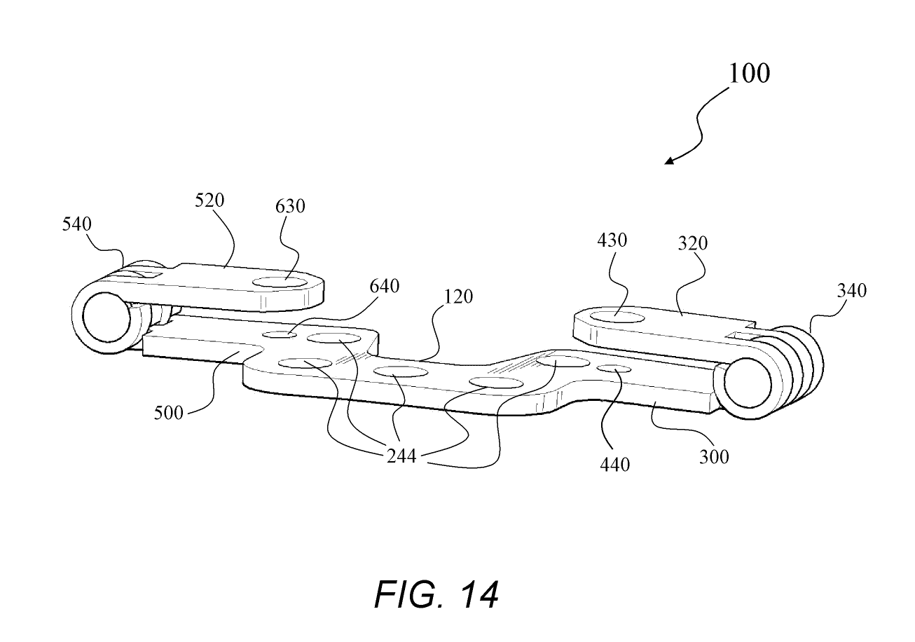

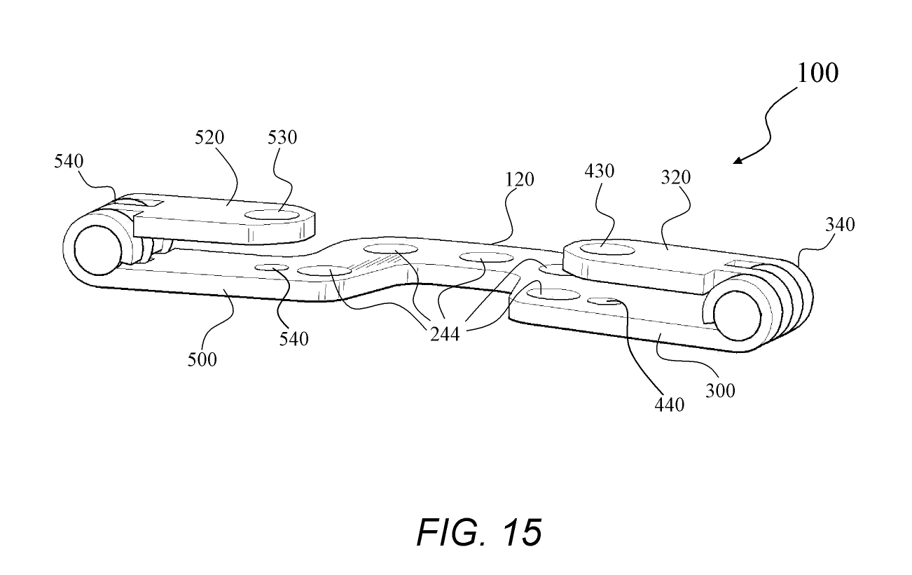

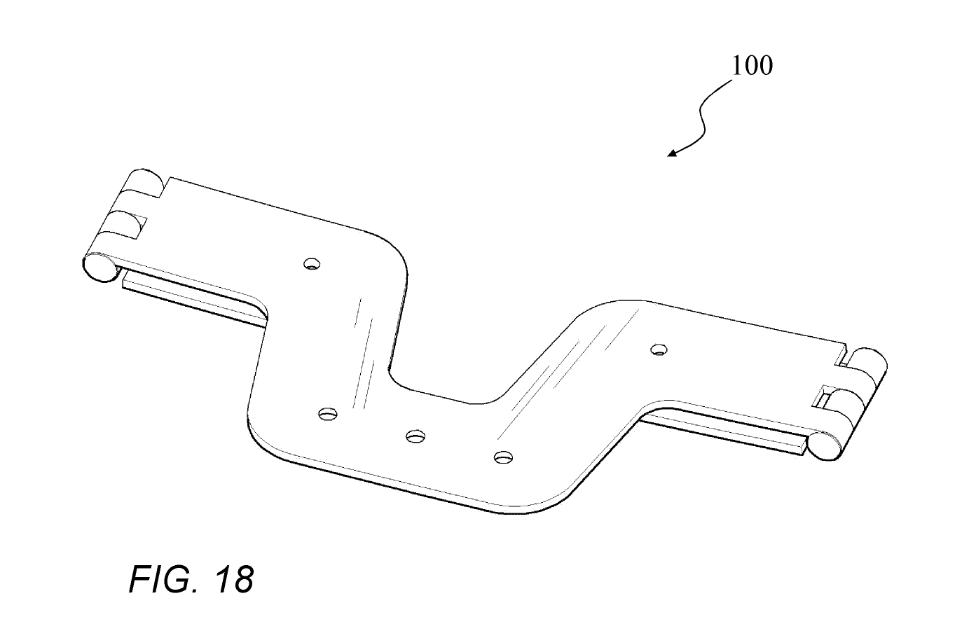

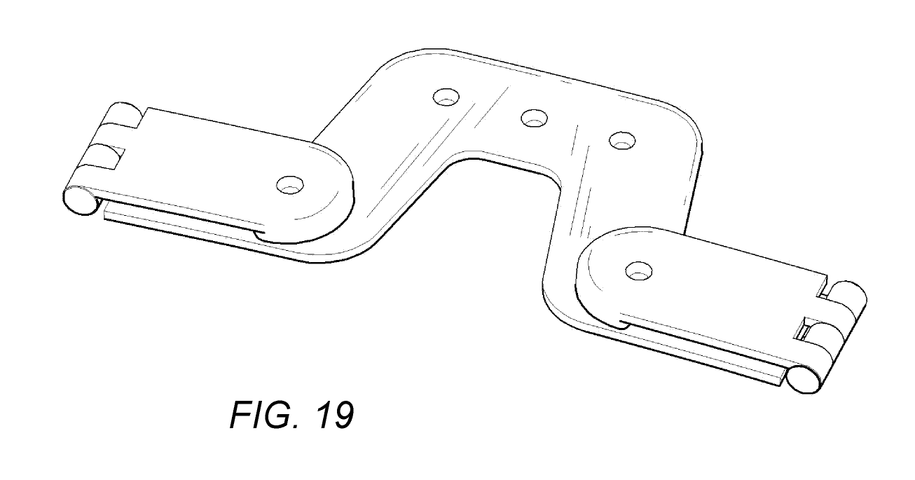

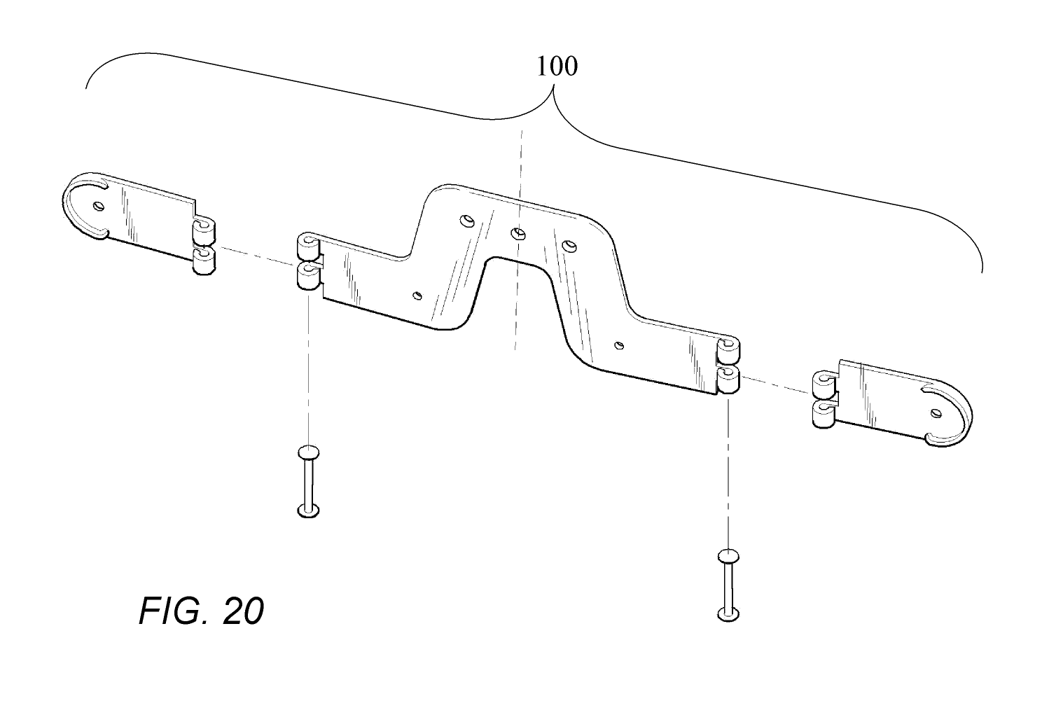

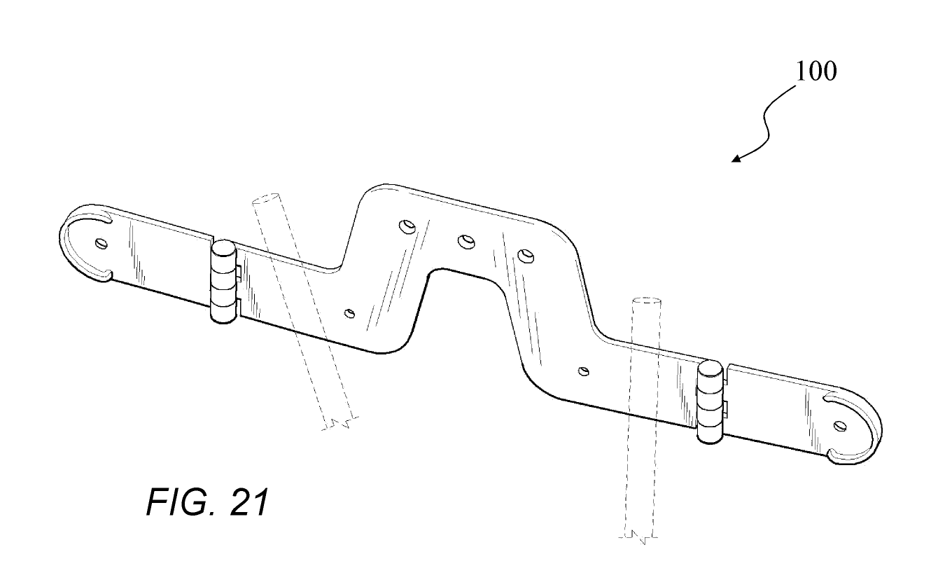

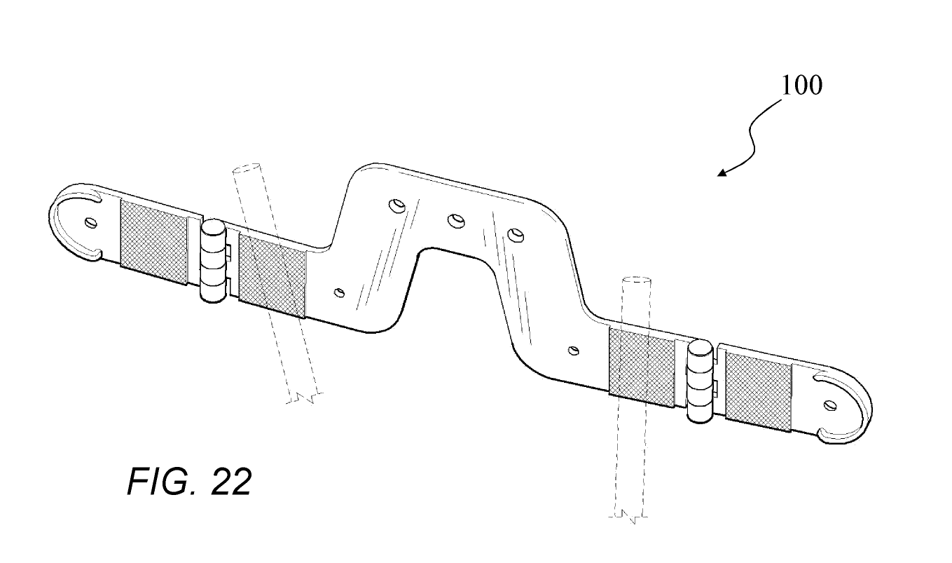

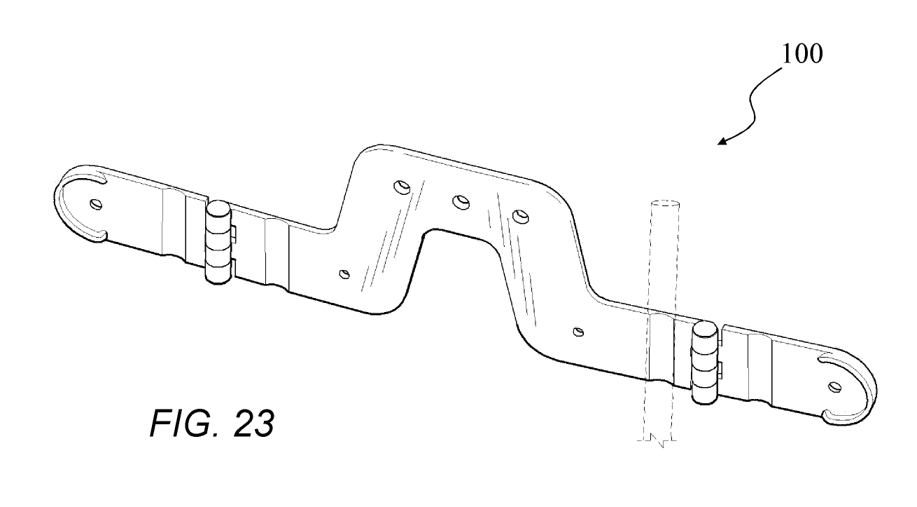

Attention is now directed to FIGS. 1-23, wherein the meaning of labels and numbers shown in the Figures is summarized in Table 1 (see, FIG. 16).

FIG. 1 illustrates an environmental perspective view of an occipital plate, according to an embodiment. The occipital plate 100 according to aspects of the embodiments is shown attached by fasteners 225 to the lower posterior curve of the skull known as the occipital region Oc.

FIGS. 2, 3 and 4 respectively illustrate a front view of the occipital plate 100 of FIG. 1, but with left and right hinged legs 140 and 160 deployed in an open, a partially closed, and a closed configuration, respectively. In this embodiment, the left and right fastener receiving holes 460 and 660 are located in the middle portion 120 of occipital plate 100.

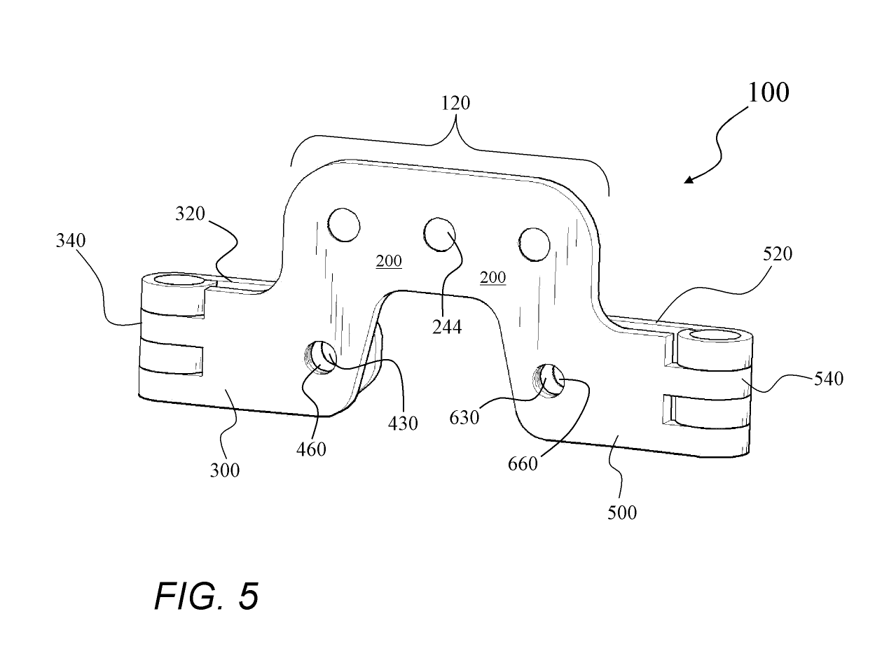

FIGS. 5, 6, and 7 respectively illustrate rear, top side, and bottom side views of the occipital plate 100 shown in closed configuration. The holes 430 and 460 are shown aligned, and likewise holes 630 and 660 are aligned and each pair of holes are able to receive a fastener to secure spinal rods SR1 and SR2 (not shown) to the right and left hinged legs 140 and 160, respectively.

FIG. 8 illustrates a front view of an occipital plate with first and second hinged legs 140 and 160 with high friction front surfaces according to an embodiment. According to a further embodiment, surfaces 344, 346, 544 and 546 can be machined to offer high friction surfaces to secure rods SR1 and SR2 (not shown).

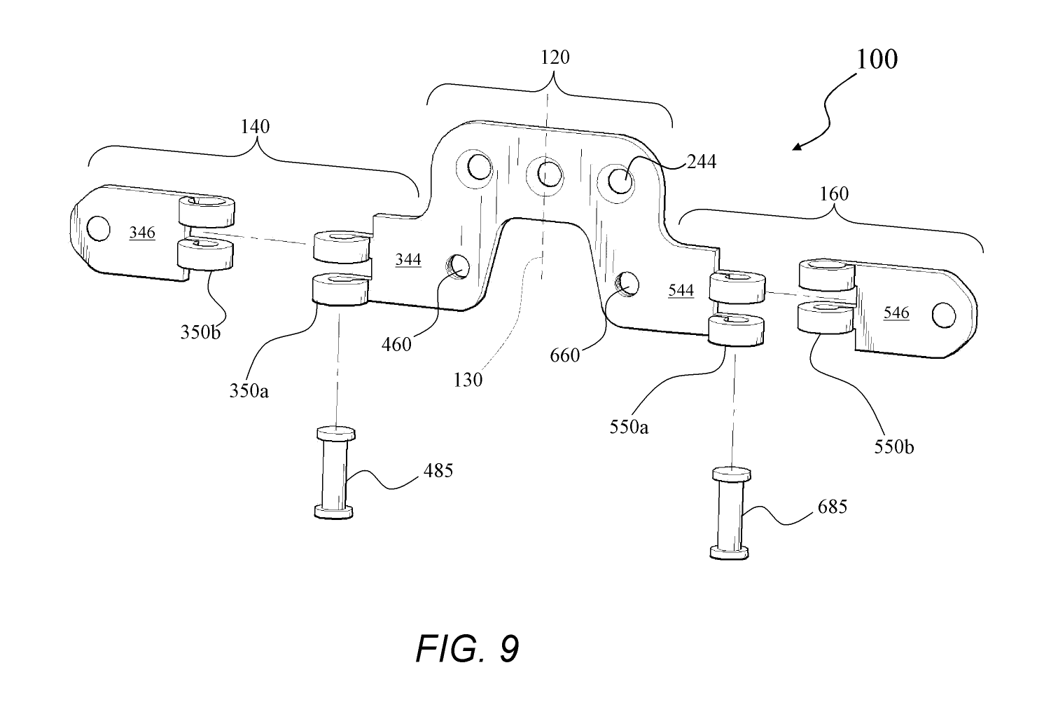

FIG. 9 illustrates an exploded view of the occipital plate 100 of FIG. 1, according to an embodiment. Left and right hinge pins 485 and 685 are shown therein. It should be understood by those of skill in the art that any suitable hinge mechanism can be used and is not limited to that shown in FIG. 9.

FIG. 10 illustrates an occipital plate 100, according to an embodiment, wherein the proximal end 360 of left stationary leg portion 300 defines a left fastener receiving hole 440, and the proximal end 560 of right stationary leg portion 500 defines a right fastener receiving hole 640. The fastener receiving holes 440 and 640 can be threaded blind holes. According to this embodiment, a fastener, such as a locking screw (not shown), can be used to fasten the distal end 420 of the left rotatable leg portion 320 to the proximal end 360 of stationary leg portion 300, and a fastener, such as a locking screw (not shown), can be used to fasten the distal end 620 of the right rotatable leg portion 320 to the proximal end 560 of stationary leg portion 500. As those of skill in the art can further appreciate, such fasteners are not limited to screws, as many other types of fasteners can be used, including, for example, bio-medical glues, staples, rivets, nut and bolt assemblies, among others.

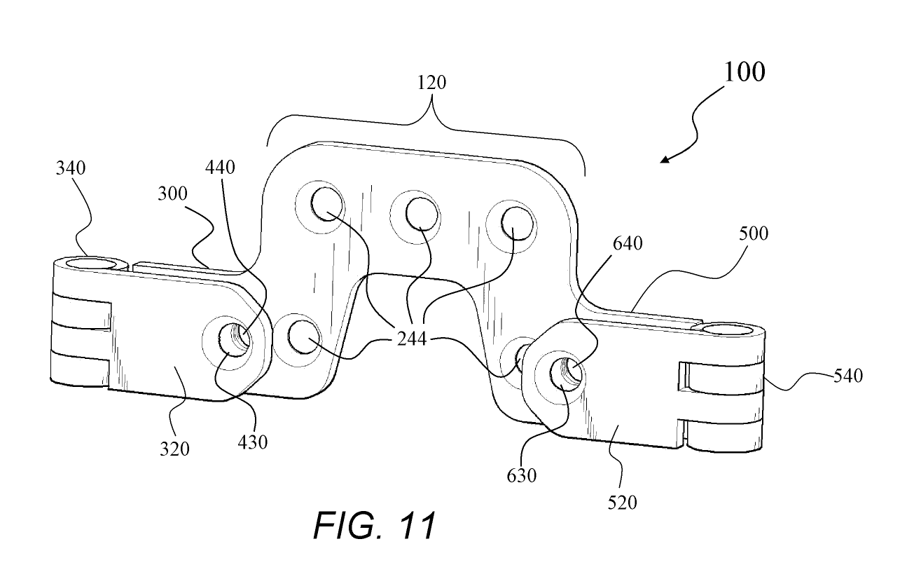

FIGS. 11 through 15 illustrate various views of the occipital plate 100 as shown in FIG. 10 according to an embodiment. As discussed above, FIG. 16 illustrates Table 1, which comprises a list of part numbers used in FIGS. 1-15 and 17-23.

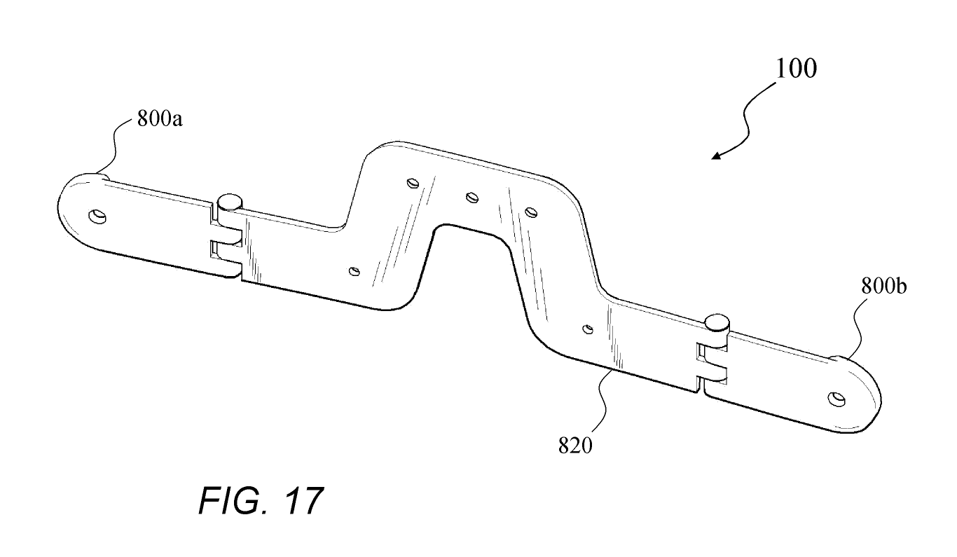

According to an embodiment, occipital plate 100 includes at least one curved perimeter portion 800 (e.g. see, FIG. 17 wherein the at least one curved perimeter portion 800 is represented by labels 800a and 800b). According to a further embodiment, occipital plate 100 defines a perimeter 820, which further defines said at least one curved perimeter portion 800. The at least one curved perimeter portion 800 serves to help smooth selected edges of the occipital plate 100. During actual use of this embodiment of occipital plate 100, the at least one curved perimeter portion 800 helps avoid snagging of a patient's tissue, such as muscle tissue, on the occipital plate 100.

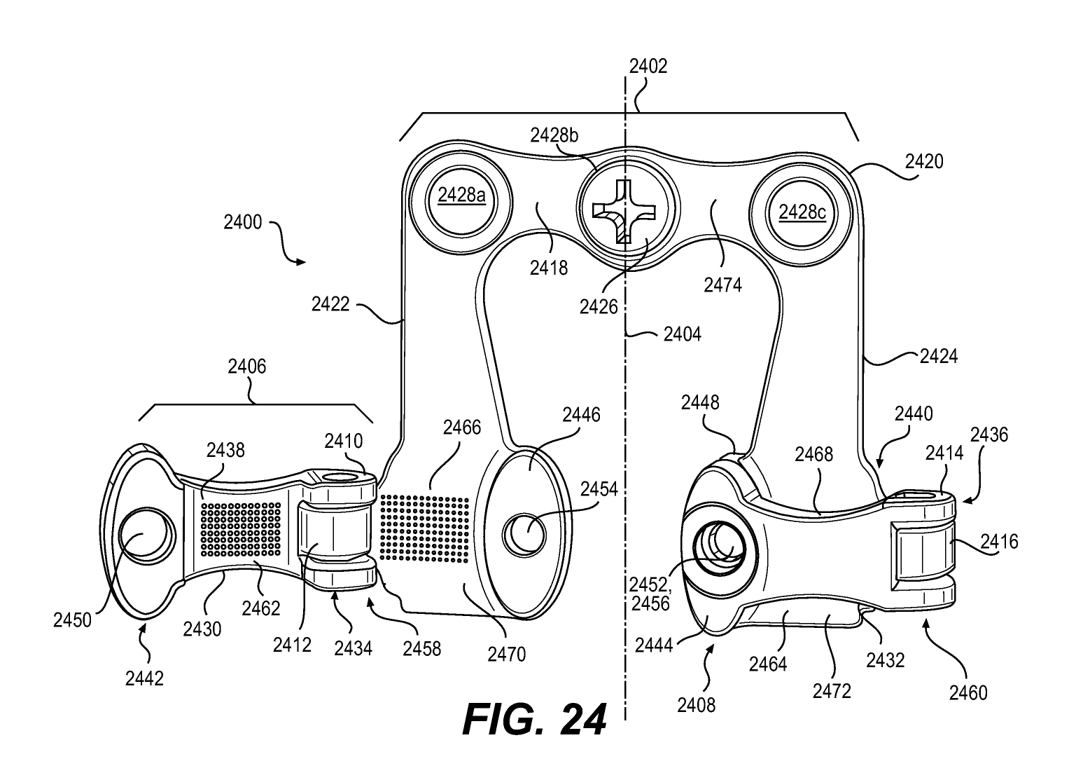

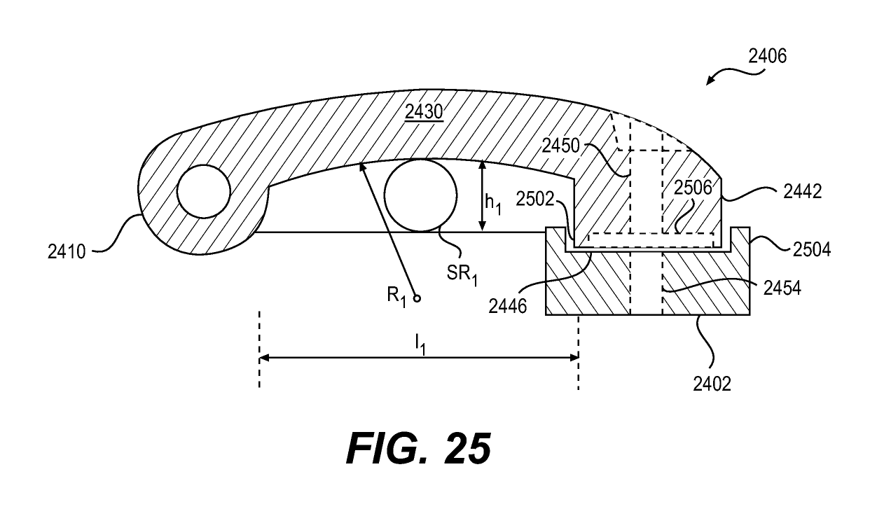

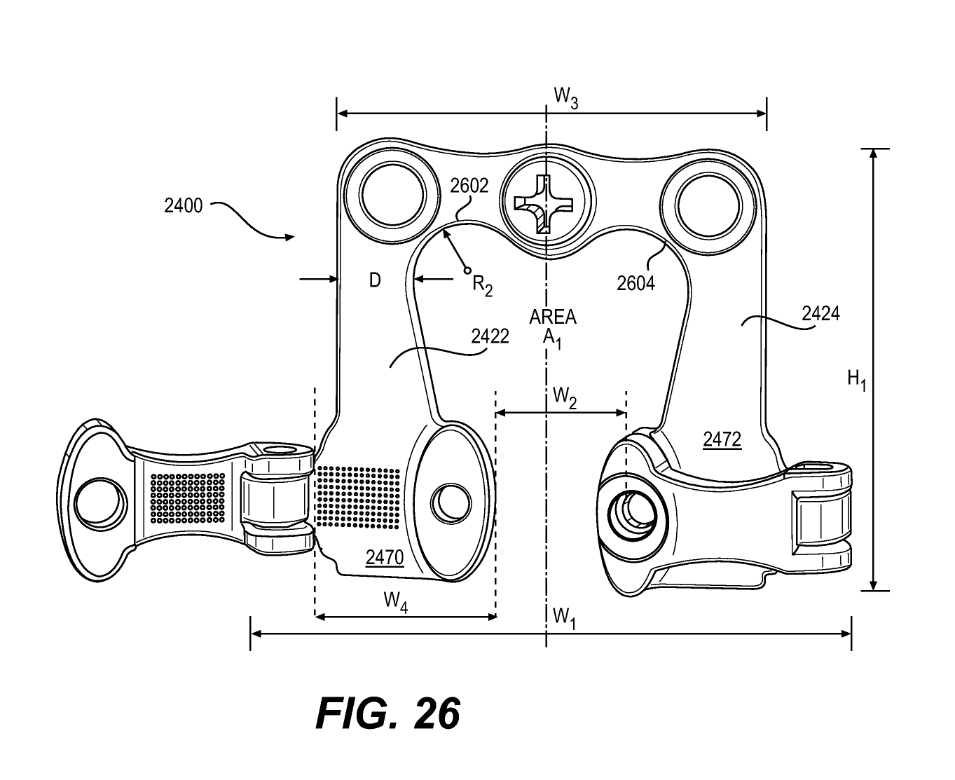

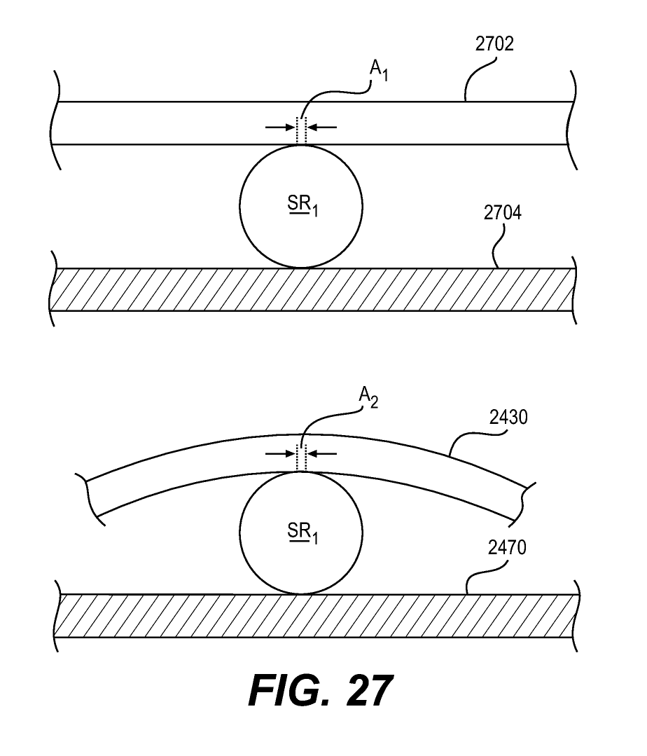

Attention is now directed towards FIGS. 24-27. FIG. 24 illustrates a front perspective view of occipital plate 2400 with left and right rounded locking hinges 2406, 2408 according to an embodiment, FIG. 25 illustrates a close-up side view of left rounded locking hinge 2406 and left leg oval shaped receptacle 2446 according to an embodiment, FIG. 26 illustrates a further front perspective view of occipital plate 2400 according to an embodiment, and FIG. 27 illustrates a side view of conventional (non-curved) rotatable leg portion 2702 when securing a first spinal rod against a bottom portion 2704 of an occipital plate, and a side view of left rotatable leg portion 2430 according to an embodiment securing a first spinal rod against bottom portion 2470 of occipital plate 2400 of FIG. 24 according to an embodiment.

Within these drawings, substantially similar features are included therein with respect to occipital plate 2400 as were included and discussed above in regard to occipital plate 100; however, in fulfillment of the dual purposes of clarity and brevity, a detailed discussion of the same features has been omitted. Instead, occipital plate 2400 includes at least several additional features according to further embodiments that are now discussed in regard to FIGS. 24-28. Unless otherwise mentioned or specifically discussed herein, occipital plate 2400 incorporates similar features as was discussed in regard to occipital plate 100.

According to an embodiment, occipital plate 2400 includes several main components: middle portion 2402, central axis line 2404, left rounded locking hinge 2406, and right rounded locking hinge 2408. Each of these main components themselves contain components, each of which will be discussed in turn. Middle portion 2402 includes top member 2474, front surface 2418, rear surface 2420 (rear surface 2420 is the surface that contacts the skull of the patient that uses occipital plate 2400), left member 2422, and right member 2424. Left member 2422 includes left bottom member 2470, and right member 2424 includes right bottom member 2472. Generally, top member 2474 includes the part of middle portion 2402 that extends from and includes the area from and around first mounting hole 2428a to area at and about second mounting hole 2428b. Left member 2422 of middle portion 2402 includes that portion that extends down from a bottom portion of first mounting hole 2428a through to the bottommost portion of left bottom member 2470 including left leg oval shaped receptacle 2446, and right member 2424 of middle portion 2402, extends from a bottom portion of second mounting hole 2428b through to the bottommost portion of right bottom member 2472 including right leg oval shaped receptacle 2448.

Left rounded locking hinge 2406 of occipital plate 2400 includes left leg hinge component 2410, which, in conjunction with middle portion left leg hinge component 2412 and left hinge pin 2458, form left hinge joint 2434, and which provides for the hinging action of left rotatable leg portion 2430 that forms left rounded locking hinge 2406. Left rotatable leg portion 2430 includes front surface 2438, upon which is formed left leg gripping surface 2462. Left leg gripping surface 2462 can be a plurality of raised portions, commonly referred to as a “knurled surface,” and the knurls can be of any shape, including, but not limited to, spherical, diamond shaped, among others, or the plurality of raised portions can be a plurality of ridges (both relatively straight, or formed in a curved or wavy pattern), among other types of raised surfaces, all of which are considered to be within the scope of the embodiments. Left bottom member 2470 includes middle portion left side gripping surface 2466. Left side gripping surface 2466 can be a plurality of raised portions, commonly referred to as a “knurled surface,” and the knurls can be of any shape, including, but not limited to, spherical, diamond shaped, among others, or the plurality of raised portions can be a plurality of ridges (both relatively straight, or formed in a curved or wavy pattern), among other types of raised surfaces, all of which are considered to be within the scope of the embodiments.

Left rotatable leg portion 2430 can further include left leg oval shaped portion 2442, through which is formed left leg fastener hole 2450. Left leg fastener hole 2450 is generally not threaded, though that need not necessarily be the case, and it works with left fastener receiving hole 2454 that is part of middle portion 2402, and more particularly located within left leg oval shaped receptacle 2446. Left fastener receiving hole 2454 also need not be threaded, but it can be, in order to more securely fasten left rotatable leg portion 2430 to middle portion 2402 by fastener 2426. Additional aspects of left rotatable leg portion 2430 and left rounded locking hinge 2406 are described in greater detail in regard to FIG. 25.

Right rounded locking hinge 2408 of occipital plate 2400 includes right leg hinge component 2414, which, in conjunction with middle portion right leg hinge component 2416 and right hinge pin 2460, form right hinge joint 2436, which provides for the hinging action of right rotatable leg portion 2432 that forms right rounded locking hinge 2408. Right rotatable leg portion 2432 includes front surface 2440, upon which is formed right leg gripping surface 2464. Right leg gripping surface 2464 can be a plurality of raised portions, commonly referred to as a “knurled surface,” and the knurls can be of any shape, including, but not limited to, spherical, diamond shaped, among others, or the plurality of raised portions can be a plurality of ridges (both relatively straight, or formed in a curved or wavy pattern), among other types of raised surfaces, all of which are considered to be within the scope of the embodiments. Right bottom member 2472 includes middle portion right side gripping surface 2468. Right side gripping surface 2468 can be a plurality of raised portions, commonly referred to as a “knurled surface,” and the knurls can be of any shape, including, but not limited to, spherical, diamond shaped, among others, or the plurality of raised portions can be a plurality of ridges (both relatively straight, or formed in a curved or wavy pattern), among other types of raised surfaces, all of which are considered to be within the scope of the embodiments.

Right rotatable leg portion 2432 can further include right leg oval shaped portion 2444, through which is formed right leg fastener hole 2452. Right leg fastener hole 2452 is generally not threaded, though that need not necessarily be the case, and it works with right fastener receiving hole 2456 that is part of middle portion 2402, and more particularly located within right leg oval shaped receptacle 2448. Right fastener receiving hole 2456 also need not be threaded, but it can be, in order to more securely fasten right rotatable leg portion 2432 to middle portion 2402 by fastener 2426. Additional aspects of right rotatable leg portion 2432 and right rounded locking hinge 2408 are substantially similar to left rotatable leg portion 2432 and left rounded locking hinge 2406 that will be described in greater detail in regard to FIG. 25 and therefore will not be discussed in fulfillment of the dual purposes of clarity and brevity.

Attention is now directed towards FIG. 25, which illustrates a close-up side view of left rounded locking hinge 2406 and left leg oval shaped receptacle 2446 according to an embodiment. As can be seen in FIG. 25, left rotatable leg portion 2430 has a generally rounded shape, with a radius of curvature r₁. As discussed in regard to FIGS. 25-28, reference is made to several dimensions, including several radii, angles, lengths or distances, among others. Those of skill in the art can appreciate that although examples of dimensions are provided, these should not be taken in a limiting manner; that is, the aspects of the embodiments are not to be construed as defined or limited by the specific example of the dimensions shown and discussed, but instead are provided merely for illustrating an example of what a device that incorporates the aspects of the embodiments could, in a non-limiting manner, look like. Furthermore, as those of skill in the art can appreciate, since the aspects of the embodiments are directed towards a physical object, with dimensional characteristics, all of the parts will have various dimensions, some of which are not shown in fulfillment of the dual purposes of clarity and brevity. According to still further aspects of the embodiments, some of these objects will have dimensional characteristics that lend themselves to aesthetic aspects; in fulfillment of the dual purposes of clarity and brevity, discussion of the dimensions in this regard have also been omitted. Therefore, as the aspects of the embodiments are directed towards a occipital plate used in surgery, it is to be understood that the dimensions of the different objects, some dimensions shown, some dimensions not shown, will be understood by those of skill in the art.

According to an embodiment, therefore, r₁ can be chosen to be a radius that accommodates typically used spinal rods, SR₁ and SR₂. As those of skill in the art can further appreciate, the radius r₁ can depend on the length shown in FIG. 25 of l₁, the height h₁ (which is dependent upon the diameter of spinal rods SR₁ and SR₂). By imparting a radius of curvature onto left rotatable leg portion 2430, several features are realized according different aspects of the embodiments. First, the curved shape of left rotatable leg portion 2430 makes it less likely to snag on, or tear into the patient's internal musculature and other body parts. That is, when occipital plate 2400 according to an embodiment is implanted into a patient to fasten and secure their neck into a known position, the body tissue that covers occipital plate 2400 will be substantially less likely to find an edge to get snagged on, thereby making use of occipital plate 2400 much more comfortable to the user (patient) than prior art devices. Furthermore, according to another embodiment, by making left rotatable leg portion 2430 with a radius of curvature, more surface area of front surface area of left rotatable leg portion, including more surface area of left leg gripping surface 2462, will come into contact with the spinal rod, SR₁. Since a greater proportion of left leg gripping surface 2462 comes into contact with SR₁, more gripping power can be imparted onto SR₁ thereby making it substantially more likely to keep SR₁ in place and keep it from slipping. As those of skill in the art can appreciate, it is rarely the case where additional strength does not come at the cost of additional comfort; in this case according to an embodiment, additional strength goes hand-in-hand with the additional comfort provided by the radius of curvature imparted into left rotatable leg portion 2430.

Further shown in FIG. 25 is the dimension h₁, which describes the height wherein the spinal rode SR₁ traverses left rotatable leg portion 2430. Although it is generally preferred that SR₁ traverse left rotatable leg portion 2430 approximately at the middle of left rotatable leg portion 2430, as seen in FIG. 25, this need not be the case. That is, a sufficient amount of flexibility has be designed into left rotatable leg portion 2430 to accommodate less than ideal placement of SR₁. The height h₁ is incorporated by design through the length of left rotatable leg portion 2430, l₁, the radius of curvature r₁, the height of left leg oval shaped distal protrusion 2442 and the depth of left leg oval shaped receptacle 2446.

As those of skill in the art can further appreciate, although spinal rods SR₁ and SR₂ have been described as being substantially cylindrical in shaped, that need not necessarily be the case. For example, according to further embodiments, the spinal rods can exhibit a rectangular or square or oval or triangular cross shape. According to further embodiments, the spinal rod can include cross sectional shape characteristics that include two or more of the shapes discussed above, as well as others not mentioned, all of which are considered to be within the aspects of the embodiments.

According to further embodiments, additional strengthening or securing features have been designed into occipital plate 100. As briefly described above, left rotatable leg portion 2430 also includes left leg oval shaped distal protrusion 2442; as those of skill in the art can appreciate, the embodiments described herein are not meant to be, and should not be so construed to be limited to an “oval” shape; other shapes can be used, including, but not limited to circular, rectangular, squares, among others. Left leg oval shaped distal protrusion 2442 is designed to fit into left leg oval shaped receptacle 2446 to provide a substantially secure fit that substantially prevents any side-to-side movement in any direction. That is, because left rotatable leg portion 2430 is securely held not only by a fastener 2426 but also by a receptacle in a male-female mechanical mating relationship, the possibility of movement of left rotatable leg portion 2430 is substantially diminished, thereby making it significantly more likely that occipital plate 2400 can securely hold the skull of the patient/user in place, with little or no movement, making recovery time shorter, and with significantly less discomfort. Left leg protrusion extension 2502 is a lip shaped protrusion located at a lowermost portion of left leg oval shaped distal protrusion 2442, that forms left leg protrusion recess 2506. As those of skill in the art can appreciate, left leg oval shaped distal protrusion 2442 need not include left leg protrusion extension 2502 and left leg protrusion recess 2506; instead, the bottommost portion of left leg oval shaped distal protrusion 2442 could be substantially planar, or it too could have a knurled surface that could interact with a knurled surface on a receiving surface of left leg oval shaped receptacle 2446 according to an embodiment. Left leg oval shaped receptacle 2446 is formed by middle portion left side receptacle lip 2504 as shown in FIG. 25 according to an embodiment. As those of skill in the art can appreciate, the shape of the receptacle of middle portion 2402 generally needs to match that of the distal protrusion of left rotatable leg portion 2430.

Attention is now directed towards FIG. 26, which illustrates a further front perspective view of occipital plate 2400 according to an embodiment. In FIG. 26, which is substantially similar to FIG. 24, several dimensions of occipital plate 2400 are shown and will be discussed. As is readily apparent from FIGS. 24-26, occipital plate 2400 does not include a stationary leg portion as does occipital plate 100 and other prior art devices. By eliminating a stationary leg portion, the surface area of occipital plate 2400 has been reduced; that is, occipital plate 2400 now covers less surface area of the skull and spinal area of the patient, yet provides a substantially secure manner of removably fixing the skull of the patient in place in relationship to the spine of the patient. Thus, the overall width W of occipital plate 2400 can be smaller than that of prior art devices. It can be appreciated by those of skill in the art, however, that such reduction in covered surface area does not come at the price of reduced strength, because the bottom portion 2470, 2472 of both the left and right members 2422, 2424 are wider than their counterpart portions of occipital plate 100, and other prior art devices, in order to provide the torque strength needed to secure left and right rounded locking hinges 2406, 2408 to the skull of the patient and to a middle portion 2402 of occipital plate 2400. Thus, width dimension W₁ is greater than width dimension W₄. According to an embodiment, and as those of skill in the art can appreciate (and as discussed above in regard to the dimensions discussed in regard to FIG. 25, among others), such dimensions are not to be construed in a limiting sense, but just as one example of an aspect of the embodiments, as many different sizes of occipital plate 2400 are possible, W₁ can be about 50 mm, and can range from about 30 mm to about 70 mm; according to a further embodiment, W₄ can be about 15 mm, and can range from about 10 mm to about 20 mm. According to further aspects of the embodiments, D can be about 5 mm, and can range from about 3 mm to about 7 mm, W₂ can be about 12 mm and can range from about 10 mm to about 14 mm; W₃ can be about 35 mm and can range from about 30 mm to about 40 mm; and H can be about 34 mm and can range from about 30 mm to about 38 mm.

As further shown in FIG. 26, occipital plate 2400 includes left and right rounded internal corners 2602, 2604. Each of left and right rounded internal corners 2602, 2604 incorporate a second radius of curvature r₂ into an interior area A₁ of occipital plate 2400 in furtherance of at least two reasons. First, rounding the internal corners 2602, 2604 distributes any stress that might be built up in that area to be distributed more readily, and substantially eliminates the possibility of stress fracture in that area. Stresses can occur as a result of torque that occurs as a result of the forces imparted onto middle portion 2402 of occipital plate 2400 due to first and second spinal rods, SR₁ and SR₂, particularly in the area of left bottom member 2470 and right bottom member 2472, which is then transferred to upper portions of left member of middle portion 2422, and right member of middle portion 2424, respectively.

In addition to re-distributing stress in the area, left and right rounded internal corners 2602, 2604 decrease the amount of metal at the top portion 2474 of middle portion 2402 of occipital plate 2400. This decreases the amount of surface area that occipital plate 2400 covers of the skull of the patient. That is, occipital plate 2400 has been designed to minimally impact the patient by lessening the surface area of the skull and spine that is touched or covered by occipital plate 2400 to only that which is necessary to impart the strength for securing the skull in place in the desired relationship with the spine (through use of spinal rods SR₁ and SR₂). Or, more simply put, occipital plate 2400 does not touch or impact any more bone of the patient than it needs to.

In furtherance of the desire to lessen any negative impact of use of occipital plate 2400, as can be seen in especially FIGS. 24 and 26, substantially all of the edges of occipital plate are beveled or rounded. The rounding of all edges, combined with the radius of curvature that is designed into left rotatable leg portion 2430 and right rotatable leg portion 2432 means that there is substantially no sharp or 90° corners of metal of occipital plate 2400 that can catch or hook into the tissue of the patient. This allows the muscle and other tissue to slide relatively freely over occipital plate 2400 after it has been implanted.

According to an embodiment, although not shown in any of the aforementioned FIGS., occipital plate 2400 has a thickness t of about 2 mm. According to further aspects of the embodiments, thickness t can range from about 1 mm to about 3 mm, or can even be thicker, depending on the circumstances of the injury sustained by the patient, or thinner, for the same reason. According to a further aspect of the embodiments, the rounding of all of, or substantially all of the edges of occipital plate 2400 can be about 0.75 mm, or can range from about 0.5 mm to about 1.0 mm. Therefore, according to still further aspects of the embodiments, a ratio of the radius of curvature (r_{c}), for rounding of the edges of occipital plate 2400 to the thickness t of occipital plate 2400 can be about 37.5% (r_{c}/t), and can range from about 25% to about 50%. As those of skill in the art can appreciate, the radius of curvature for rounding, r_{c}, can further depend on the size and thickness of occipital plate 2400, the material it is made of, among other factors, all of which are considered to be within the aspects of the embodiments.

FIG. 27 illustrates a side view of conventional (non-curved) rotatable leg portion 2702 when securing a first spinal rod against a bottom portion 2704 of an occipital plate, and a side view of left rotatable leg portion 2430 according to an embodiment securing a first spinal rod against left bottom member 2470 of occipital plate 2400 of FIG. 24 according to an embodiment. As briefly discussed above, left rotatable leg portion 2430, because of the radius of curvature imparted into it, will increase the surface area between itself and spinal rode SR₁ according to an embodiment, which increase the amount of securing force against SR₁. As shown in FIG. 27, the amount of surface area between conventional (i.e., non-curved) rotatable leg portion 2702 and first spinal rod is smaller than that between left rotatable leg portion 2430 and spinal rod SR₁. As those of skill in the art can appreciate, the same relationship exists for right rotatable leg portion 2432, second spinal rod SR₂, and right bottom member 2472 of occipital plate 2400 according to an embodiment.

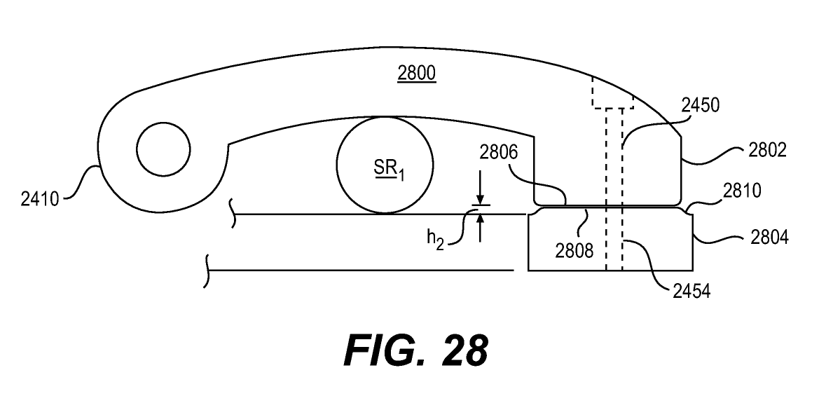

FIG. 28 illustrates a close-up side view of a left side rounded locking hinge and receiving portion of occipital plate 2400 according to a further embodiment. As can be seen in regard to FIG. 28, left rotating leg portion 2800, according to an embodiment, is similar in form to left rotating leg portion 2403 as shown in FIG. 25, but with the exception that left rotating leg portion 2800 includes left leg distal protrusion 2802 that is of a different design than that of left leg oval shaped distal protrusion 2442 as shown in FIG. 25. As such, left rotating leg portion 2800 includes the same features and characteristics of a radius r₁, l₁, and h₁, although not shown in FIG. 28. Further, middle portion of occipital plate 2402 now includes left leg protrusion receiving portion 2804 that correspondingly mates with left leg distal protrusion 2802 according to an embodiment. Left leg protrusion receiving portion 2804 is similarly located on middle portion of occipital plate 2402 as that of left leg oval shaped receptacle 2446 as shown in FIG. 25. As further shown in FIG. 28, left rotating leg portion 2800 includes left leg fastener hole 2450, and left leg protrusion receiving portion 2804 includes left fastener receiving hole 2504

While left rotating leg portion 2800 is similar in shape to left rotating leg portion 2403, there are differences according to a further embodiment. For example, left rotating leg portion 2800 includes at its distal position left leg distal protrusion 2802, which, while according to an embodiment is oval shaped (but not necessarily limited in shape thereto, as discussed above), has a substantially flat left leg protrusion surface 2806. Left leg protrusion surface 2806 is substantially similar in terms of shape and surface characteristics to that of left leg protrusion receiving surface 2808 located on middle portion of occipital plate 2402. According to an embodiment, the surfaces of both left leg protrusion surface 2806 and left leg protrusion receiving surface 2808 can both be substantially planar, i.e., smooth and flat, or both can be knurled (as other surfaces have been described above), or one or the other can be substantially planar and the other knurled. According to still a further embodiment, the “knurled” surface of left leg protrusion surface 2806 can mate with the “knurled” surface of left leg receiving portion 2808 so that a knurled protrusion on one surface can match a knurled recess on an opposite surface, and visa-versa.

According to a further embodiment, both left rotating leg portion 2800 (and its counterpart, right rotating leg portion, which is not shown in FIG. 28, or others, but which is substantially similar to as left rotating leg portion 2800) incorporates the rounded edges for substantially similar reasons as discussed above. That is, all or substantially all of the edges are rounded to reduce and/or substantially eliminate the possibility of snagging on tissue of the user of occipital plate 2400. Thus, left leg protrusion receiving surface 2808 further includes left leg protrusion receiving portion fillet edge 2810, as shown in FIG. 28; left leg protrusion receiving portion fillet edge 2810 would, according to an embodiment, completely encompass left leg protrusion receiving portion 2804, and have a height h₂, as indicated in FIG. 28.

Although not shown, those of skill in the art can appreciate that occipital plate 2400 further includes a right rotating leg portion, a right leg distal protrusion, a right leg protrusion receiving portion, right leg protrusion surface (which can be knurled, as described above in regard to left leg protrusion surface 2806), right leg protrusion receiving surface (which can be knurled, as described above with regard to left leg protrusion receiving surface 2808), and a right leg protrusion receiving portion fillet edge, all of which can be substantially similar (or dissimilar) in characteristics as their left leg counterparts, according to an embodiment, as one of skill in the art can appreciate.

Although the features and elements of the embodiments are described in the embodiments in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and can include other examples that occur to those skilled in the art. Such other examples are intended to be within the scope of the claims.

The above-described embodiments are intended to be illustrative in all respects, rather than restrictive, of the embodiments. Thus the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

I claim:

1. An occipital plate comprising:
a middle portion having opposite left and right sides;
a left rounded locking hinge for receiving and securing a first spinal rod (SRA wherein the left rounded locking hinge extends from the left side of the middle portion, and wherein
the left rounded locking hinge includes
a left leg middle portion that is concavely curved with respect to a left side substantially flat surface of the left side of the middle portion, and
a left leg distal protrusion located at a distal end of the left leg middle portion; and
a right rounded locking hinge for receiving and securing a second spinal rod (SR2), wherein the right rounded locking hinge extends from the right side of the middle portion, and wherein
the right rounded locking hinge includes
a right leg middle portion that is concavely curved with respect to a right side substantially flat surface of the right side of the middle portion, and
a right leg distal protrusion located at a distal end of the right leg middle portion.

2. The occipital plate according to claim 1, wherein, the middle portion further comprises:
a left side shaped receptacle adapted to receive the left leg distal protrusion located at the distal end of the left leg middle portion when the left rounded locking hinge is rotated to secure the first spinal rod; and
a right side shaped receptacle adapted to receive the right leg distal protrusion located at the distal end of the right leg middle portion, when the right rounded locking hinge is rotated to secure the second spinal rod.

3. The occipital plate according to claim 2, wherein
the left leg distal protrusion, the right leg distal protrusion, the left side shaped receptacle, and the right side shaped receptacle are shaped according to a shape that can include at least one of an oval, a circle, a square, triangle, and a rectangle.

4. The occipital plate according to claim 2, wherein
the left shaped receptacle includes a middle portion left side receptacle lip adapted to retain the left leg distal protrusion, and
the right shaped receptacle includes a middle portion right side receptacle lip adapted to retain the right leg distal protrusion.

5. The occipital plate according to claim 1 further comprising:
a left side gripping surface located on the left side substantially flat surface adapted to grip said first spinal rod;
a right side gripping surface located on the right side substantially flat surface adapted to grip said second spinal rod;
a left rounded locking hinge gripping surface located on a surface of the left rounded locking hinge opposite to that of the left side gripping surface when in the securing position, and adapted to grip said first spinal rod; and
a right rounded locking hinge gripping surface located on a surface of the right rounded locking hinge opposite to that of the right side gripping surface when in the securing position, and adapted to grip said second spinal rod.

6. The occipital plate according to claim 1, wherein the middle portion comprises:
a left member;
a right member; and
a top member, wherein said top member extends from an uppermost portion of the left member to an uppermost position of the right member.

7. The occipital plate according to claim 1, wherein
substantially all edges are rounded.

8. The occipital plate according to claim 7, wherein a radius of curvature for the rounding of substantially all of the edges ranges from about 0.5 mm to about 1.0 mm.

9. The occipital plate according to claim 1, wherein, the middle portion further comprises:
a left leg protrusion receiving portion adapted to receive the left leg distal protrusion located at the distal end of the left leg middle portion when the left rounded locking hinge is rotated to secure the first spinal rod; and
a right leg protrusion receiving portion adapted to receive the right leg distal protrusion located at the distal end of the right leg middle portion, when the right rounded locking hinge is rotated to secure the second spinal rod.

10. The occipital plate according to claim 9, wherein
the left leg distal protrusion, the right leg distal protrusion, the left leg protrusion receiving portion, and the right leg protrusion receiving portion are shaped according to a shape that can include at least one of an oval, a circle, a square, triangle, and a rectangle.

11. The occipital plate according to claim 9, wherein
the left leg distal protrusion includes a substantially planar left leg protrusion surface,
the left leg protrusion receiving portion includes a substantially planar left leg protrusion receiving surface adapted to retain the left leg distal protrusion,
the right leg distal protrusion includes a substantially planar right leg protrusion surface, and
the right leg protrusion receiving portion includes a substantially planar right leg protrusion receiving surface adapted to retain the left leg distal protrusion.

12. The occipital plate according to claim 11, wherein
the substantially planar left leg protrusion surface and the substantially planar left leg protrusion receiving surface can both be substantially smooth.

13. The occipital plate according to claim 11, wherein
the substantially planar left leg protrusion surface and the substantially planar left leg protrusion receiving surface can both include a knurled surface.

14. The occipital plate according to claim 11, wherein
one of the substantially planar left leg protrusion surface and the substantially planar left leg protrusion receiving surface can include a knurled surface.

15. The occipital plate according to claim 11, wherein
the substantially planar right leg protrusion surface and the substantially planar right leg protrusion receiving surface can both be substantially smooth.

16. The occipital plate according to claim 11, wherein
the substantially planar right leg protrusion surface and the substantially planar right leg protrusion receiving surface can both include a knurled surface.

17. The occipital plate according to claim 11, wherein
one of the substantially planar right leg protrusion surface and the substantially planar right leg protrusion receiving surface can include a knurled surface.

18. The occipital plate according to claim 9, wherein the left leg protrusion receiving portion comprises:
a left leg protrusion receiving portion fillet edge with a height hi extending above an upper surface of the middle portion.

19. The occipital plate according to claim 9, wherein the right leg protrusion receiving portion comprises:
a right leg protrusion receiving portion fillet edge with a height hi extending above an upper surface of the middle portion.

20. An occipital plate comprising:
a middle portion having opposite left and right sides;
a left hinged leg for receiving and securing a first spinal rod, wherein the left hinged leg extends from the left side of the middle portion;
a right hinged leg for receiving and securing a second spinal rod, wherein the right hinged leg extends from the right side of the middle portion; and
a left leg curved protrusion located at a distal portion of the left hinged leg; and
a right leg curved protrusion located at a distal portion of the right hinged leg.
